(19) United States
(12) Patent Application Publication
(10) Pub. No.: US 2024/0258818 A1
(43) Pub. Date: Aug. 1, 2024

(54) CHARGING SYSTEM FOR A RECHARGEABLE IMPLANTABLE STIMULATION SYSTEM

(71) Applicant: Axonics, Inc., Irvine, CA (US)

(72) Inventors: Faizal ABDEEN, Mission Viejo, CA (US); Xuechen HUANG, Irvine, CA (US); Guangqiang JIANG, Irvine, CA (US); Rinda SAMA, Irvine, CA (US)

(73) Assignee: Axonics, Inc., Irvine, CA (US)

(21) Appl. No.: 18/430,021

(22) Filed: Feb. 1, 2024

Related U.S. Application Data
(60) Provisional application No. 63/442,731, filed on Feb. 1, 2023.

(51) Int. Cl. H02J 7/00; H02J 50/10

(52) U.S. Cl. CPC H02J 7/007192; H02J 7/00309; H02J 7/0048; H02J 50/10

(57) ABSTRACT

A charging device for an implantable pulse generator, the device including a charging coil and a controller. The controller being configured to track a net expended thermal energy of the charging device and modulate a charging duty cycle of the charging device to limit the heat generated by the charging device based on the net expended thermal energy. The charging device is configured to charge the implantable based on the modulated charging duty cycle by providing a voltage to the charging coil.

### CROSS-REFERENCES TO RELATED APPLICATIONS

The present application claims priority to and the benefit of U.S. Provisional Patent Application No. 63/442,731, filed Feb. 1, 2023. The foregoing provisional application is incorporated by reference herein.

### BACKGROUND

This application discloses a neural-stimulation system that includes a rechargeable implantable pulse generator and an external charging device. The system also includes a heat regulation method for controlling the external charging device using duty cycle modulation and net thermal energy tracking to safely charge the implantable medical device by limiting the heat generated by the charging device.

Treatments with neurostimulation systems have become increasingly common over the last fifteen years. These neurostimulation systems generally have a neurostimulation component (for example, a pulse generator) and one or more interfacing components. The pulse generator may be an implantable pulse generator (IPG) or an external pulse generator (EPG). The interfacing components may include a charging device (CD). The charging device may be able to, for example, wirelessly charge an IPG and send or receive data from the IPG or an interfacing component.

While neurostimulation systems have been widely implemented in treating a number of conditions, there are still a number of implementation problems that need to be addressed. For example, charging devices may operate non-optimally or may pose safety risks when they are subjected to excessive temperatures that may result from a combination of the environment in which they are used and heat produced by the charging devices themselves. Thus, it may be advantageous to devise methods, systems, and devices for monitoring and regulating temperatures of charging devices while they are in use so as to ensure optimal safety and efficacy. Given the effects of neurostimulation systems on patient health and the attending safety risks associated with these systems, it may be particularly desirable to monitor and regulate these systems.

### SUMMARY

This application discloses a neural-stimulation system that includes a rechargeable implantable pulse generator and an external charging device. The system also includes a heat regulation method for controlling the external charging device using duty cycle modulation and net thermal energy tracking to safely charge the implantable medical device. Further, this application discloses an embodiment wherein the heat regulation method operates without the need for thermal sensors.

The disclosed implantable neural-stimulation system may be used for sacral nerve stimulation (SNM) for the treatment of overactive bladder (OAB). The implantable components of the rechargeable SNM system include a rechargeable Implantable Pulse Generator (IPG) and a lead. The typical location of the IPG and lead in the body is shown in FIG. 1.

The external charging device (CD) disclosed herein may include a heat regulation method. The CD also may include a battery, circuitry for generating the electromagnetic field for charging the IPG, a controller and device to facilitate the operation of the CD and storage of a control system for the method and other data. The method or control system for the CD may include a duty cycle modulation scheme such that the CD can operate at desired limits for heat generation. The method or control system may further include a tracking of net expended thermal energy in heat units such that the desired limit for heat generation is determined as a function of net expended thermal energy.

In one embodiment, the charging circuit in the CD includes a charging coil, and when charging an IPG, the CD produces an amplitude of voltage or current in the coil to drive the charging process such that the IPG rectified charge voltage is maintained in its desired operating range. In one embodiment, the CD controller monitors and measures the power drawn from the battery and determines an operating duty cycle such that the rate of heat generation by the CD is maintained below an applicable operational heat limit. In other words, the measurements and the resulting adjustments to the heat generated by the CD are based on the voltage and current (i.e., power generated) of the battery. Therefore, regardless of the inductive coupling conditions with the IPG, the CD output is duty-cycle modulated in order to maintain a CD rate of heat generation level below a preferred operating level or target. Alternately, the power drawn can be pre-characterized using a look up table and the values in the look up table can be used in lieu of or concurrently with measuring withdrawn CD battery power.

The disclosed methods control the charging operation based on the heat generated by the CD and are not based on the heat generated by the IPG and/or the heat delivered to a patient. The method does not require the use of temperature sensors for sensing the temperature of the device or for sensing the temperature of the patient.

In one embodiment, a method of operating a rechargeable system for providing neurostimulation to a patient is disclosed herein. The system including a charging device (CD) and an implantable pulse generator (IPG) and the method comprising the steps of, tracking, by a controller, a heat counter, wherein the heat counter is a value indicative of net expended thermal energy of the CD based on an operating power of the CD and a charging duty cycle of the CD, modulating, by the controller, the charging duty cycle to limit heat generated by the CD based on the heat counter; and charging the IPG based on the modulated charging duty cycle.

In one embodiment, a method of operating a charging device (CD) for an implantable pulse generator (IPG) is disclosed herein. The method comprising the steps of, tracking, by a controller, a net expended thermal energy of the CD, modulating, by the controller, a charging duty cycle to limit the heat generated by the CD based on the net expended thermal energy, and the controller controls the charging of the IPG based on the modulated charging duty cycle.

In one embodiment, a charging device (CD) for an implantable pulse generator (IPG) is disclosed herein. The CD comprises a charging coil, a battery, a controller configured to control functions of the CD, wherein the controller is configured to, track a net expended thermal energy of the CD, modulate a charging duty cycle of the CD to limit the heat generated by the CD based on the net expended thermal energy, and direct a provision of a current to the charging coil in order to charge the IPG based on the modulated charging duty cycle.

### BRIEF DESCRIPTION OF THE DRAWINGS

FIG. 1 shows an example of a neurostimulation system having an implantable stimulation lead and an implantable pulse generator.

FIG. 2 schematically illustrates an exemplary neurostimulation system including a rechargeable implantable impulse generator and a charging device.

FIG. 3 shows an example of an external charging device when not in use.

FIG. 4 shows an example of an external charging device held in a recharging position relative to an IPG.

FIG. 5 shows a flowchart illustrating one embodiment of a process for regulating the heat generation of a neurostimulation system.

FIG. 6 shows a functional block diagram for the operation of a method of charging device heat regulation.

FIG. 7 is a schematic illustration of a charging device and an implantable pulse generator.

### DETAILED DESCRIPTION

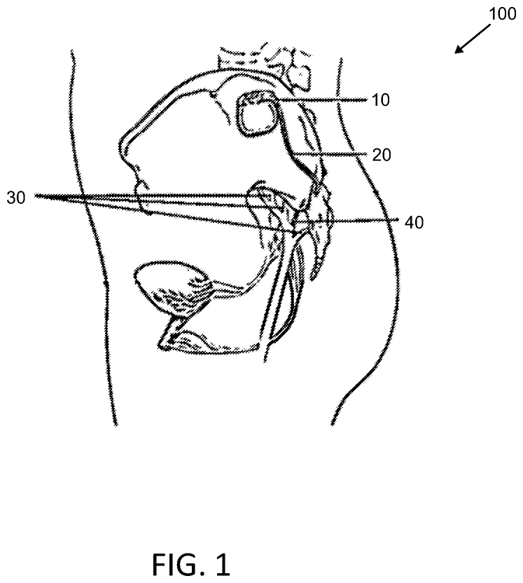

FIG. 1 shows an example of a neurostimulation system 100 having an implantable stimulation lead 20 and an implantable pulse generator 10 configured to stimulate one or more nerves 30. The typical location of the IPG 10 and lead 20 with electrodes 40 in the body is shown as an exemplary embodiment in FIG. 1.

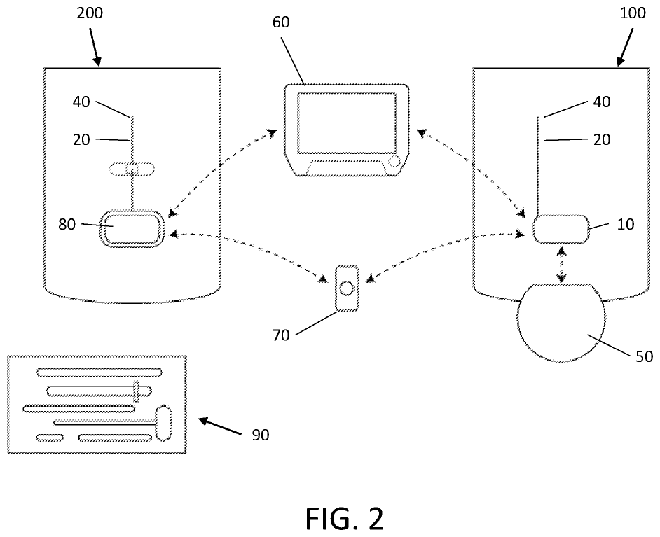

FIG. 2 schematically illustrates an exemplary neurostimulation system 100 including a rechargeable implantable impulse generator 10 and a charging device 50. In one embodiment, the rechargeable implantable impulse generator (IPG) 10 is a rechargeable implanted device that provides electrical pulses to stimulate a target sacral nerve.

In one embodiment, the neurostimulation system 100 includes a lead 20. In one embodiment, the lead 20 is a tined lead that also includes a plurality of electrode contacts 40 to carry stimulation pulses. In one embodiment, the distal tip of the lead 20 is implanted through the applicable foramen near the S3 sacral nerve with the proximal end of the lead 20 connected to the IPG 10. In one embodiment, tines on the lead 20 facilitate fixation of the lead 20 just posterior to the sacral foramen.

In one embodiment, the neurostimulation system 100 includes a remote control (RC) 70. In one embodiment, the RC 70 is a non-rechargeable battery powered device that uses radio-frequency (RF) signals to communicate with the IPG 10. In one embodiment, the RC 70 allows the subject to check and adjust the stimulation level, to check the status of the IPG 10 battery charge level and to turn stimulation on or off.

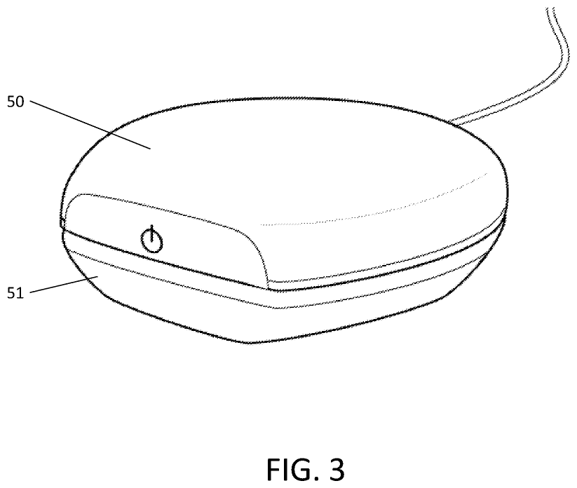

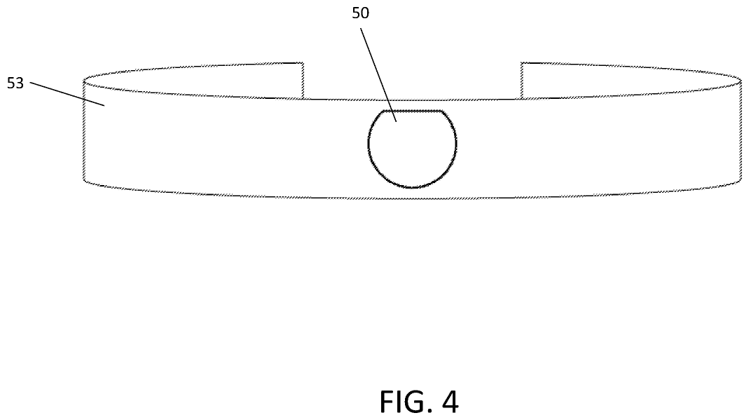

In one embodiment, the neurostimulation system 100 includes a charging device (CD) 50. In one embodiment, the CD 50 is an external portable device powered by a rechargeable battery. The CD 50 may be configured for transcutaneous charging of the IPG 10 through electromagnetic induction. In one embodiment, as seen in FIG. 3, the external CD 50 is compatible with a dock 51 that is configured to connect to a wall outlet and may be used to recharge the external CD 50. The CD 50 can be either patched to the patient's skin using an adhesive or can be held in place using a belt 53 (as seen in FIG. 4) or by an adhesive patch.

In one embodiment, the neurostimulation system 100 includes a clinician programmer (CP) 60, which may be embodied as a tablet computer used by a clinician to program the neurostimulation system 100. In one embodiment, the CP 60 communicates wirelessly with the IPG 10.

In one embodiment, the neurostimulation system 100 is designed to work in tandem with an external trial system 200. The external trial system 200 may be used prior to permanent implantation of the neurostimulation system 100 to screen a patient for SNM therapy. In one embodiment, the external trial system 200 includes an external pulse generator (EPG) 80 that connects to the lead 20 in place of the IPG 10 and interfaces with both the RC 70 and the CP 60 in a manner similar to the IPG 10.

In order to implant the IPG 10, a suite of surgical tools 90 may be necessary. The suite of surgical tools 90 may include a foramen needle with needle stylet, a directional guide, an introducer sheath and dilator, a lead stylet (with straight or curved tip), a torque wrench, a tunneling tool, a needle stimulation cable, and a lead stimulation cable.

FIG. 3 shows an example of an external charging device 50 when not in use. In one embodiment, the CD 50 is an external portable accessory of the SNM System. In FIG. 3, the charging device 50 is resting on a charging dock 51 that can be used to recharge the charging device 50.

FIG. 4 shows an example of an external charging device 50 held in a recharging position relative to an IPG 10 (not depicted in FIG. 4). In one embodiment, the CD 50 allows a patient to recharge the IPG 10 wirelessly through electromagnetic induction. When the CD 50 is placed on the patient's skin over the IPG 10, the CD 50 will start charging the IPG 10 battery. As shown in FIG. 4, a belt 53 or a carrier may be used to hold the charging device or charger 50 in place while charging the IPG 10. FIG. 4 shows how a belt 53 may be used to place or adhere the CD 50 on a person's skin while the IPG 10 is recharging.

Charging an IPG 10 typically takes approximately one hour depending on the depth of the IPG 10 and how the CD 50 is positioned relative to the IPG 10. For efficient charging, the CD 50 should be placed directly over the IPG 10 at the correct position, which provides the optimal alignment between the CD 50 and IPG 10. When the alignment between the CD 50 and IPG 10 is suboptimal, charging is inefficient, thereby generating heat in the charging device. To ensure safe charging of deep implants or when the CD 50 and IPG 10 are misaligned, it is necessary to operate the CD 50 in such a manner to regulate the heat generated in the charging device.

In one embodiment, a heat limit operating profile W_{OHL} is defined such that the operation of the CD cannot lead to the heat generated by charging the IPG exceeding the heat limit operating profile W_{OHL}. The control of the heat generation can be accomplished through the use of a controller (i.e., a processor) within the CD. The controller may be configured to execute instructions that are stored in a memory device located in the CD.

The heat limit operating profile W_{OHL} can be varied depending on the state of the CD. For example, heat limit operating profile W_{OHL} is determined such that when the CD is known to be cool, faster charging is allowed at an enhanced heat generation wattage Wₕᵢ. Conversely, when the CD is potentially warm, (i.e., when thermal energy approaches a maximum value Jₘₐₓ) operation transitions to a reduced heat generation wattage limit Wₗₒ. In one embodiment, Jₘₐₓ, Wₕᵢ and Wₗₒ are determined by empirical characterization.

The CD heat regulation method includes the use of an internal counter to track the amount of net thermal energy J drawn from the CD battery. In one embodiment, the counter increments J as a value measuring thermal energy in a unit of milli-Joules that is stored inside a non-volatile/non-transitory memory located in the CD.

In one embodiment, the counter increments J to track the net energy expended while power is produced by the battery of the CD (i.e., the CD power). In one embodiment, the counter can decrement J to model device cool down in an inactive mode. In one embodiment, inactive modes may include power off mode and charging dock powered mode.

Because the rate of CD's internal heat generation due to the charging process is a portion of the CD power produced by the CD's battery, measuring the latter (CD peak power: Wₚₑₐₖ) is an appropriate means for regulating the former. By modulating the duty cycle D such that the CD average power W=D×Wₚₑₐₖ≤W_{OHL}, the CD can regulate the rate heat generation and therefore maintain the CD temperature within an acceptable operating level. As evident by the aforementioned equation, it should be noted that W_{OHL} is an upper limiting condition, thus the average operating power of the CD may be equal to or lower than the operational heat generation limit W_{OHL}.

The disclosed method limits the CD average power W by measuring the voltage and current that is output by the CD battery, and using the voltage and current to compute Wₚₑₐₖ. Alternatively, voltage and power measurement can also be performed at the coil power generation stage. Alternatively, the method employs a lookup table to determine power Wₚₑₐₖ based on either or both of the voltage or current produced by the CD battery.

The CD heat generation limit W_{OHL} is determined as a function of heat units counter value J as follows (Table 1).

TABLE 1

| Operational | Heat units | CD Operational Heat |
|---|---|---|
| Range | counter range | Generation Limit |
| Low Heat Limit | <Jₘᵢₙ | W_{OHL} = Wₕᵢ |
| High Heat Limit | ≥Jₘₐₓ | W_{OHL} = Wₗₒ, where |
| | | W_{OHL} = W_{lo, depleted} if charge |
| | | C < C_{depleted} |
| | | W_{OHL} = W_{lo, charged} otherwise |
| Intermediate | Jₘᵢₙ-Jₘₐₓ | Wₕᵢ > W_{OHL} > Wₗₒ |
| Heat Limit | | |

Table 1 shows exemplary operational heat generation limits for the charging device as a function of the net expended thermal energy.

When the heat counter is greater than or equal to Jₘₐₓ, the operational heat generation limit W_{OHL} will be set to a reduced amount Wₗₒ. If sufficient charge C (e.g., remaining energy capacity in the battery measured in mAh) exists in the CD battery, then Wₗₒ=W_{lo,charged}. The controller may be further configured to check whether the charge C of the CD is less than a set minimum charge value C_{depleted} such that if the battery of the CD is less than the set minimum C_{depleted}, then Wₗₒ=W_{lo,depleted}. This depleted operational heat generation limit is even lower than the normal charged (i.e., W_{lo,depleted}<W_{lo,charged}) such that the battery of the CD is protected at lower states of charge.

In one embodiment, when the CD is off or is powered by the charging dock (i.e., when not charging the IPG), the accumulated heat unit J decrements at a preset rate as a function of the heat units counter's value as shown in Table 2 below.

TABLE 2

| Heat units counter range | Heat decrement rate |
|---|---|
| Jₗᵢₘ₁-Jₘₐₓ | =J_{rapid}/time |
| Jₗᵢₘ₂-Jₗᵢₘ₁ | =f(J)/time |
| <Jₗᵢₘ₂ | =J_{slow}/time |

Table 2 discloses exemplary charging device heat decrement rates and the corresponding heat units' range in the counter.

At a preset range of a high intermediate heat counter value Jₗᵢₘ₁ to a maximum heat counter value Jₘₐₓ, the heat counter will decay or decrease over time at a set rate of J_{rapid}. Thus, when the CD is on the hotter end of the heat counter range (e.g. after a long full charge session), the heat counter will decrease rapidly at a set rate of time.

At a preset range of a low intermediate heat counter value Jₗᵢₘ₂ to a a high intermediate heat counter value Jₗᵢₘ₁ the decrement rate over time of the heat counter J will be set to a function of J. This function can be a linear function or a non-linear function depending on the design of the CD.

Below a low intermediate heat counter value Jₗᵢₘ₂ the decrement rate over time of the heat counter will be set to J_{slow}. At lower heat counter values, heat will dissipate slower due to a smaller difference in the temperature of the CD and the ambient or environment. Thus, the heat decrement rate of the CD will be set at the set ranges noted above with J_{rapid}>f(J)>J_{slow}.

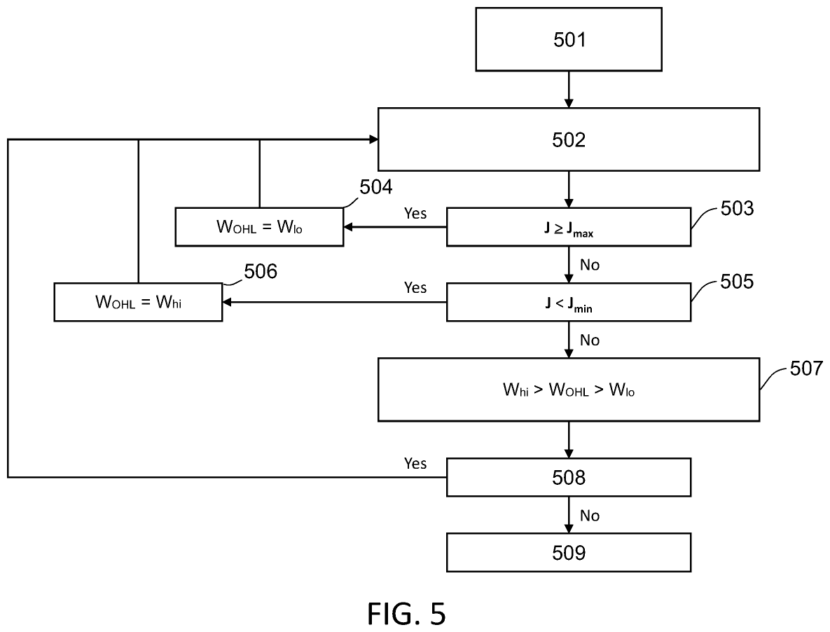

The flow chart shown in FIG. 5 provides an illustration of the control system. FIG. 5 is a flow chart that shows how the operational heat limits of the charging device are maintained according to an exemplary embodiment of the control system. The flow chart shown in FIG. 5 also shows the operational heat limits at different heat units' range.

At 501, when charging session is initiated, the CD will operate at an operational heat limit of W_{OHL}=Wₗₒ. This Wₗₒ limit may be the lowest set heat limit operating profile designed for the device.

At 502, the heat unit counter beings to track a heat unit counter J which indicative of accumulated thermal energy for the charging session. The baseline thermal energy J₀ may begin at 0, a set baseline number, or a non-baseline number if the charger was initiated before the tracked thermal energy J has completely reduced to 0. When charging is first initiated the controller will set J=J₀. At this step, the tracking of the heat counter J may further include summation of the heat counter Jₙ, which is a value indicative of the peak power of the CD weighted by the charging duty cycle of the CD at time (or increment) n+1 such that J=J₀+ΣJₙ, where n can be different times or number of increments set at temporal increments (or any set increments) n+1 by the controller to track, collect, and/or sum the heat counter J at time (or number of increments) n. Since the heat counter J relies upon a modulated duty cycle of the CD, J will vary in value. At higher duty cycles, a larger heat counter is added while a lower duty cycle will result in a smaller heat counter being added to the previously tracked heat counter.

At step 503 the controller will check if the unit heat counter J is greater than or equal to Jₘₐₓ. When the heat counter J is greater than or equal to Jₘₐₓ, the operational heat generation limit W_{OHL} will be set to a reduced amount Wₗₒ, that is, the controller will modulate the charging duty cycle such that the target operational heat generation limit to be Wₗₒ at step 504. The controller may be further configured to check whether the charge C of the CD is less than a set minimum charge value C_{depleted} such that if the battery of the CD is less than the set minimum C_{depleted}, then Wₗₒ=W_{lo,depleted} at step 504. This depleted operational heat generation limit is even lower than the normal charged (i.e. W_{lo,depleted}<W_{lo,charged}) such that the battery of the CD is protected at lower states of charge to prevent damage. If sufficient charge C is contained in the CD battery, then Wₗₒ=W_{lo,charged} at step 504. Following step 504, the controller will return to step 502 and continue tracking the heat counter J after some time (or increment number) n, at 502. Typically, when the heat counter J reaches to or above Jₘₐₓ, the device will continuously run at a high heat operational range where W_{OHL} will be set to a reduced amount Wₗₒ until the charging session is finished. It should be noted that W_{OHL} is an upper limiting condition, thus the actual operating power of the CD may be equal to or lower than the operational heat generation limit W_{OHL}.

At step 505, if heat counter J≥Jₘₐₓ is not true, then the controller will check for if J<Jₘᵢₙ at this step. If the condition J<Jₘᵢₙ is true, this means that CD is currently at a lower temperature operating range and have a higher thermal capacity such that W_{OHL} may be equal to an enhanced charging Wₕᵢ at step 506. At W_{OHL}=Wₕᵢ the CD is allowed its maximum operational power as long as the condition J<Jₘᵢₙ is true. Following step 506, the controller will return to step 502 and continue tracking the heat counter J after some time (or increment number) n.

At step 507, if both conditions J≥Jₘₐₓ and J<Jₘᵢₙ are both not true, then the operational heat generation limit W_{OHL} will be limited between Wₕᵢ and Wₗₒ. At this step, W_{OHL} is determined as a function of heat units counter value J, such that W_{OHL}=f(J), where f(J) can be linear or non-linear. In one exemplary embodiment f(J) can be a linear interpolation between Wₕᵢ and Wₗₒ. In one exemplary embodiment, f(J) can be numerically retrieved by the controller from a look up table saved within the memory of the CD.

At step 508, the controller may be configured check if the CD is still charging the IPG. If the CD is no longer charging the IPG, at step 509, the controller will start decrementing heat counter J per unit time (or per increment) based on the amount set on Table 2. If the CD is on, step 502 will be repeated and continue tracking the heat counter J after some time (or increment number) n.

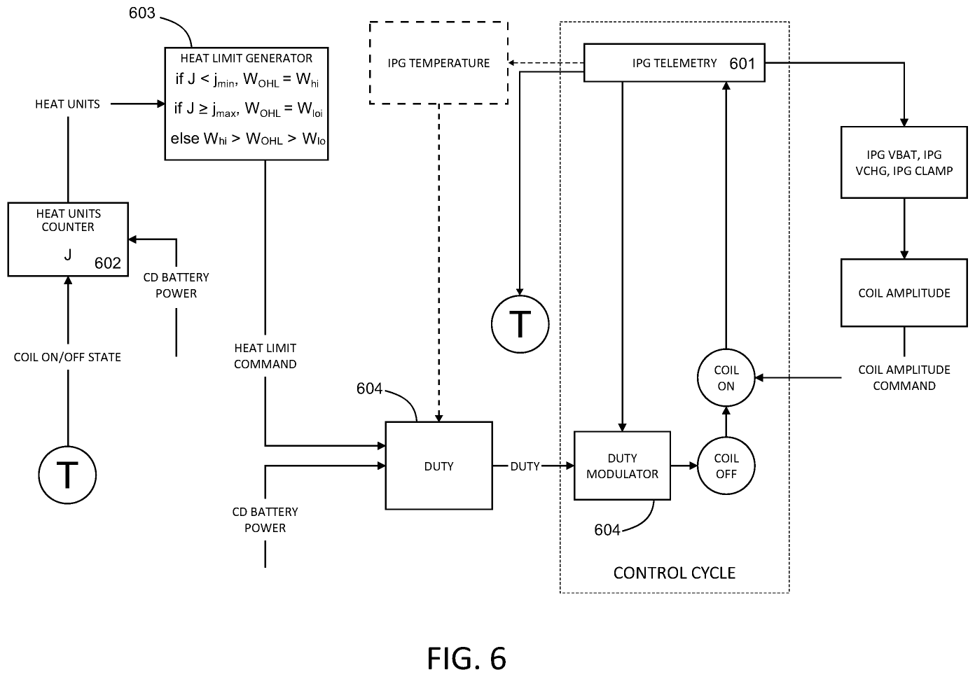

FIG. 6 shows a functional block diagram of the controller of the CD for the operation of a method of charging device heat regulation. As shown in FIG. 6, the duty cycle of the CD may also change due to temperature feedback from the IPG. For example, if a temperature set point associated with the IPG is exceeded, the duty cycle of the CD may be adjusted to prevent the IPG from exceeding a temperature limit. Temperature sensors may be used for monitoring the temperature of the IPG in a conventional manner. On the contrary, as described herein, the heat generated by the CD may be controlled without the use of any temperature sensors by monitoring the thermal energy drawn from the battery of the CD. As shown by the dashed lines, the IPG temperature block can be omitted and is not required for the full control of the heat regulation of the CD. As shown in FIG. 6, the IPG telemetry block 601 sends the coil on/off state (i.e., coil duty cycle or charging duty cycle) to the heat unit counter block 602 in order for the controller to calculate and track J in order to asses which heat limit condition to execute in block 603. The heat limit condition will be translated by control block 604 to a corresponding duty cycle limit that matches the heat limit conditions set in block 603. The control block 604 will command the duty cycle modulator block 605 such that the duty cycle limit will never exceed a set amount corresponding to the operation heat generation limits set at block 603.

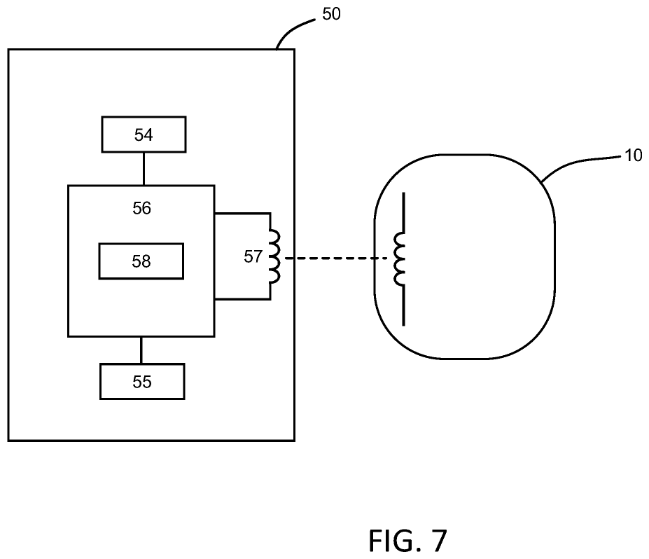

FIG. 7 shows a simplified schematic of a charging device 50 and an IPG 10 being charged. The CD 50 may include a memory 54, a power supply 55 an electronic circuit 56 configured to power the charging coil 57, and a controller 58 configured to control the electronic circuit. The power supply 55 may be a battery or a power module receiving power from an external source. Memory 54 may be any data storage device such volatile memory (e.g. random access memory (RAM)) or non-volatile memory. The controller 58 include a processor. Memory 54 may be a part of and located within the controller 58 (e.g. cache memory).

What is claimed is:

1. A method of operating a rechargeable system for providing neurostimulation to a patient, the system including a charging device (CD) and an implantable pulse generator (IPG) and the method comprising the steps of:
tracking, by a controller, a heat counter, wherein the heat counter is a value indicative of net expended thermal energy of the CD based on an operating power of the CD and a charging duty cycle of the CD;
modulating, by the controller, the charging duty cycle to limit heat generated by the CD based on the heat counter; and
charging the IPG based on the modulated charging duty cycle.

2. The method of claim 1, wherein the step of modulating of the charging duty cycle to limit the heat generated by the CD is based on the operating power of the CD weighted by the charging duty cycle of the CD and not based on a sensed temperature or a temperature of the patient.

3. The method of claim 1, wherein the step of modulating of the charging duty cycle includes limiting an average operating power of the CD over a period of time to a power level at or below a heat limit operating profile.

4. The method of claim 3, wherein heat limit operating profile depends on the heat counter.

5. The method of claim 4, wherein the heat limit operating profile is at an increased value when the heat counter is below a predetermined first value; and
wherein the heat limit operating profile is at a reduced value when the heat counter is above a predetermined second value.

6. The method of claim 5, wherein the reduced value also depends on the present state of charge of a battery used to power the CD.

7. The method of claim 4, wherein the heat counter decreases over time when the CD is not charging the IPG.

8. A method of operating a charging device (CD) for an implantable pulse generator (IPG) and the method comprising the steps of:
tracking, by a controller, a net expended thermal energy of the CD;
modulating, by the controller, a charging duty cycle to limit the heat generated by the CD based on the net expended thermal energy; and
wherein the controller controls the charging of the IPG based on the modulated charging duty cycle.

9. The method of claim 8, wherein the step of modulating the charging duty cycle to limit the heat generated by the CD is based on an operating power of the CD weighted by the charging duty cycle of the CD and not based on a sensed temperature or a temperature of the patient.

10. The method of claim 8, wherein the step of modulating of the charging duty cycle limits an average operating power of the CD over a period of time to a power level at or below a heat limit operating profile.

11. The method of claim 10, wherein heat limit operating profile depends on a heat counter, wherein the heat counter is a value indicative of net expended thermal energy of the CD based on an operating power of the CD and a charging duty cycle of the CD.

12. The method of claim 11, wherein the heat limit operating profile is at an increased value when the heat counter is below a predetermined first value; and
wherein the heat limit operating profile is at a reduced value when the heat counter is above a predetermined second value.

13. The method of claim 12, wherein the reduced value also depends on the present state of present state of charge of a battery used to power the CD.

14. The method of claim 4, wherein the heat counter decreases over time when the CD is not charging the IPG.

15. A charging device (CD) for an implantable pulse generator (IPG), wherein the CD comprises:
a charging coil;
a battery;
a controller;
wherein the controller is configured to:
track a net expended thermal energy of the CD; and
modulate a charging duty cycle of the CD to limit the heat generated by the CD based on the net expended thermal energy;
direct a provision of a current to the charging coil in order to charge the IPG based on the modulated charging duty cycle.

16. The device of claim 15, wherein the CD does not include a temperature sensor.

17. The device of claim 15, wherein the controller is configured to track the net expended thermal energy by monitoring of the power drawn from the battery of the charging device.

18. The device of claim 15, wherein the controller is configured to track the net expended thermal energy of the CD utilizing a decrement rate when the CD is not charging the IPG.

19. The device of claim 15, wherein the controller is configured to modulate the charging duty cycle of the CD by changing an average power of the CD to one of at least three different power levels.

20. The device of claim 19, wherein the average power level of the CD is lower when the battery voltage of the CD is below a set charge value.
